(19) United States
(12) Patent Application Publication
(10) Pub. No.: US 2009/0155358 A1
(43) Pub. Date: Jun. 18, 2009

(54) PHARMACEUTICAL COMPOSITIONS OF SHORT-ACTING HYPNOTIC AGENTS IN MODIFIED-RELEASE FORMS AND THE PROCEDURES TO PREPARE THE MENTIONED FORMULATION

(75) Inventors: Liliana Elisabeth Diaz, Buenos Aires (AR); Gustavo Alejandro Andrade, Buenos Aires (AR)

Correspondence Address: PATENT CENTRAL LLC;Stephan A. Pendorf, 1401 Hollywood Boulevard, Hollywood, FL, 33020, US

(73) Assignee: Gador S.A., Buenos Aires (AR)

(21) Appl. No.: 12/096,700

(22) PCT Filed: Dec. 5, 2006
(86) PCT No.: PCT/EP2006/011636

§ 371 Date: Nov. 5, 2008

(30) Foreign Application Priority Data
Dec. 7, 2005 (AR) P20050105132

(51) Int. Cl. A61K 9/52; A61K 31/437; A61K 9/24

(52) U.S. Cl. 424/457; 514/300; 424/472

(57) ABSTRACT

This application refers to a modified-release pharmaceutical composition containing, as the active agent, a short-acting hypnotic agent or a pharmaceutically acceptable salt thereof, comprising two sustained-release pharmaceutical entities, differentiated from each other by a different release rate of the active agent wherein the release of the active agent from one of the entities starts before the release of the active agent from the second entity.

This application refers to stable and modified-release pharmaceutical compositions of an active ingredient with pharmaceutical activity, such as the short-acting hypnotic agents, as for instance zaleplon, zopiclone or its enantiomers as the (S or R)-zopiclone, triazolam, temazepam, brotizolam, alimemazine, indiplon and Zolpidem and latter one's tartrate or some of its pharmaceutically acceptable salts. Also to the procedures to prepare such pharmaceutical compositions.

### PRIOR ART

For many orally administered active agents, it is preferred that the molecules are released on a constant basis, or at least at a controlled speed in a determined lapse of time, as for instance 4 to 8 hours or more. The main object of the controlled release systems is to allow safety and to provide a sustained action of the therapeutic effect. Nowadays, the controlled release systems are designed to produce a more reliable absorption and to improve the bioavailability and efficiency of the active agent's delivery.

This invention's most preferred active agent is an appropriate short-acting hypnotic agent known as Zolpidem. Its name according to IUPAC is N,N,6-trimethyl-2-p-toyl-imidazo[1,2-a]pyridine-3-acetamide as tartrate salt (2:1) and which base structure is as follows:

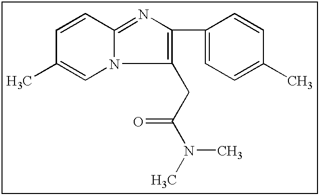

The Zolpidem tartrate is a solid white to off-white crystalline powder, sparingly soluble in water, alcohol and propylene glycol. Its molecular weight is 764.88.

Its chemical structure is unrelated to the one of benzodiazepines, barbiturates, pyrrolepirazines, pyrazolopirimidines or other drugs with known hypnotic properties. Contrasting the benzodiazepines which bind and become active in a non selective form all the subtypes of BZ receptors, the Zolpidem binds in vitro to the BZ₁ receptor, presenting a large affinity for the subunits α₁/α₅.

The controlled release preparations of Zolpidem for daily administration are considered of advantage as regards the immediate release forms available on the pharmaceutical market today, as the dose may be controlled and it can improve the patient's tolerance.

Some formulations of controlled release short-acting hypnotic agents have been found in the previous art, i.e.:

The U.S. Pat. No. 4,824,678 (Aktiebolaget Leo) which date of publication is Apr. 25, 1989 describes an oral pharmaceutical preparation with controlled release, having a biphasic profile of the active ingredient, comprising a nucleus which contains the active ingredient and a coating applied to this one, where the coating consists of a film-forming polymer insoluble in water and in the gastrointestinal fluids and a pores-forming material soluble in water which also includes the active ingredient.

The patent GB 2245492 (Zambon Group S.p.A.) which date of publication is Jan. 8, 1992 describes an oral pharmaceutical preparation with scheduled release (understand release after a predetermined delay) comprising a nucleus coated with a hydrophobic material and a surfactant.

The application of the patent WO 0033835 (Sanofi/Synthelabo) which publication date is Jun. 15, 2000 refers to controlled dosage forms for short-acting hypnotics with a diphase dissolution profile. There is also the application of the patent WO 0100181 (Sanofi/Synthelabo) which publication date is Jan. 4, 2001 referring to release forms in dual time (or what is commonly called by pulses) comprising a short-acting hypnotic agent. The first pulse being of immediate release and the second pulse is a delayed release after a certain period of time. The disadvantage of these release profiles is that, as well known, the short-acting hypnotic agents are affected by a first barrier of metabolic effect where the drug is rapidly metabolized into inactive metabolites. Using the controlled release profile defined in Sanofi-Synthelabo's application of diphase profile, the drug's bioavailability may by decreased as it presents a constant relative speed.

According to this, there exists a need in the technique's condition for hypnotic-sedative compositions to induce and maintain the sleep as simple use nightly formulations, without the presence of the adverse effects associated to long acting hypnotic agents. This invention complies with these requirements, providing further related advantages.

### OBJECT OF THE INVENTION

This application refers to modified-release pharmaceutical compositions characterized because the active agent's release which is a short-acting hypnotic agent or some of its pharmaceutically accepted salts appears as from two sustained-release pharmaceutical entities, differentiating from each other because they have a different release velocity of the active and where the active's release as from one of them starts before the release as from the second one.

The preferred short-acting hypnotic agents are zaleplon, zopiclona or its enantiomers as the R or S-zopiclona, triazolam, temazepam, brotizolam, alimemazina, indiplon and Zolpidem. Among them, the one mostly preferred is Zolpidem.

Zaleplon means N-[3-(3-cyanopyrazol[1,5-a]pyrimidine-7-il)phenyl]-N-ethylacetamide, or its pharmaceutical acceptable salts.

Zopiclone has a denomination according to IUPAC 6-(5-chlorine-2-pyridinil)-6,7-dihydro-7-oxo-5H-pyrrole[3,4-b]pyrazine-5-il-1-piperazinecarboxylate, or its pharmaceutical acceptable salts.

Triazolam means 8-chlorine-6-(o-chlorophenyl)-1-methyl-4H-s-triazol-(4,3-alpha)(1,4-benzodiazepine, or its pharmaceutical acceptable salts.

Temazepan has a denomination according to IUPAC 7-chlorine-1,3-dihydro-3-hydroxy-1-methyl-5-phenyl-2H-1,4-benzodiazepine-2-ona, or its pharmaceutical acceptable salts.

Brotizolam means 2-bromo-4-(o-chlorophenyl)-9-methyl-6H-thieno[3,2-f]-s-triazol[4,3-a][1,4]diazepine, or its pharmaceutical acceptable salts.

Alimemazina has a denomination according to IUPAC N,N-dimethyl-2-[(phenotiazine-10-il)methyl]propilamine as hemitartrate, or some of its pharmaceutical acceptable salts.

Indiplon means N-methyl-N-[3-[3-(2-thienylcarbonyl)pyrazol[1,5-alpha]pyrimidine-7-il]phenyl]acetamide, or its pharmaceutical acceptable salts.

The term “short-acting hypnotics” used in this application, refers to compounds able to induce sedative, anxiolytic, myorelaxant, and anticonvulsant effects in those mammalians to which they are administered. This application's short-acting hypnotics, although not limited to these, include pyrazolopirimidines (such as zaleplon and indiplon), cyclopyrrolones (such as zopiclone), benzodiazepines (such as triazolam, temazepam, and brotizolam), phenothiazines (such as alimemazine) and imidazopiridines (such as Zolpidem).

Advantageous forms of this invention comprise pharmaceutical compositions formed by two sustained-release entities where the matrix-forming agent contained in both entities, helpfully allows to adjust the active's release speed in a very easy way, through the concentration of the said matrix-forming agent (more concentration, less velocity) and of the use of soluble and insoluble diluents (for the presence of insoluble agents, less velocity has been found).

The mentioned pharmaceutical compositions, in particularly advantageous forms of this invention, are tablets obtained by means of a press-coating process, where the nucleus corresponds to the entity of slower sustained-release, and the outer layer corresponds to the faster sustained-release entity.

Surprisingly, thanks to an appropriate choice of the excipients and even in the case that this nucleus should be recovered by one or more polymeric coatings (which works against the formation of bindings during the application of the outer layer through compression), we have found that thanks to the choice of the excipients of the outer layer, pharmaceutical formulations are obtained having excellent pharmacotechnic characteristics of friability and hardness, which may be submitted to later coating processes (for instance, with cosmetic purposes, or to mask bitter tastes). The appropriate choice of the excipients of the composition of this invention allows to obtain nucleus as well as to apply the outer coating by direct compression, but they also may be obtained by means of other known methods, such as the wet granulation or double compression.

Besides, a further object of this invention is the procedure to prepare the mentioned pharmaceutical compositions, and the compositions obtained through this procedure.

### Advantages of the Composition

In this invention a modified-release pharmaceutical product has been obtained, satisfying the induction of the sleep and allows to preserve this therapeutic effect over a longer time.

The formulation of this invention minimizes the unwanted gastrointestinal effects, without sacrificing the therapeutic effect (induction and preservation of the sleep), furthermore preventing the irritation of the gastroesophagus in case it is withhold in that portion of the digestive tube. A very quick dissolution could be associated to a premature exposition of the esophagus with the following risk of irritation and ulceration of the esophagus and, on the other hand it would increase the chance of contact of the active with saliva, mucus, good which could eventually affect its pharmacokinetics. Additionally, and taking the case of tablets without outer coating, the patient will not perceive the bitter taste so intensely, as the perception of the taste requires that sub-stance to be dissolved”.

Besides, with a modified-release formulation composed by two sustained-release entities it never before had been achieved to allow the induction of the sleep as well as to maintain it.

### DETAILED DESCRIPTION OF THE INVENTION

The short-acting hypnotic agents used in this invention are selected among zaleplon, zopiclone or its enantiomers such as the (R or S)-zopiclone, triazolam, temazepam, brotizolam, alimemazine, indiplon and Zolpidem. Among them the one most preferred is the Zolpidem and may comprise 2-20 mg of Zolpidem tartrate.

The composition of this application comprises two entities, i.e. one of faster sustained-release and the second one of slower sustained-release.

One preferred form of this invention is the one allowing that the faster sustained-release entity, starts releasing the active agent before the slower sustained-release entity, where the slower sustained-release entity, is found as a nucleus of a tablet obtained by press-coating or as particles (pellets, microcapsules or tablets) included in a capsule or in the matrix of a tablet.

The faster sustained-release entity may be found as an outer coating applied over a nucleus by press-coating process or as particles (pellets or tablets) within a capsule or as a matrix of a tablet which includes pellets or microcapsules.

Particularly preferred form of tablet obtained by press-coating. In these forms, the final tablet as well as the nucleus may be covered by one or more polymeric coatings. In some forms, some of the polymeric coatings applied over the nucleus is soluble at pH over 5 retarding the drug's release as from the nucleus or particles, conferring gastroresistance to the mentioned entity. In the case that in the nucleus' formulation pH regulating agents with acid characteristics should be found, the application of a subcoating prior to the gastroresistant coating has been considered of benefit in order to avoid delays in the disintegration when the middle pH is 5 or more. For the final tablet's coating, those coatings having the property of masking the taste are preferred.

In this invention, the slower sustained-release entity comprises 1-10 mg of Zolpidem tartrate. Those where the slower sustained-release entity comprises 4-6 mg of Zolpidem tartrate, or 2-4 mg Zolpidem tartrate, are preferred.

Besides, the faster sustained-release entity, has a release speed of the active agent between 3 and 10 times slower than a conventional immediate release form containing the mentioned active. Those formulations where the faster sustained-release entity comprises 1-6 mg of Zolpidem tartrate are preferred. Those where the faster sustained-release entity comprises preferably 6-10 mg of Zolpidem tartrate, or 3-5 mg of Zolpidem tartrate, are particularly object of this invention.

Another preferred form of this application consists of a pharmaceutical formulation where the nucleus or particles forming part of the slower sustained-release entity comprise at least one matrix-forming agent and do not contain a disintegrating agent, and furthermore because the coating, matrix or particles forming part of the faster extended-release entity also comprise at least one matrix-forming agent, not containing any disintegrating agent either. The matrix-forming agent present in at least one of the sustained-release entities is selected among polymeric agents, or lipidic substances and preferably, the matrix-forming agent present in the faster sustained-release entity is subject to erosion.

The polymeric matrix-forming agent or also called matrix-forming polymer may be selected among derivates of cellulose or mixtures of the polymers polyvinylacetate and polyvinylpyrrolidone. Some examples of derivates of the cellulose to be used are Methylcellulose (Methocel A), carboxymethylcellulose (Tylose C), hydroxyethylcellulose (Tylose H-Natrosol), hydroxipropylcellulose (Klucel), and hydroxipropylmethylcellulose (Methocel K, E, F), other matrix-forming agents to be used are polysaccharides (galactomans, alginates, agar-agar, gums), acrylic acid's polymers (Carbopol), lipidic matrixes (ceric or hydrophobic) formed by tri, di and monoglycerids, fat acids, fat alcohols.

The preferred matrix-forming agent is the one formed by a mixture of polyvinylacetate and polyvinylpyrrolidone, marketed by BASF as Kollidon® SR. having the following composition polyvinylacetate (PM approximately 450.000) 80%, Povidone or polyvinylpirrolidona K 30 (PM approximately 50.000) 19%, stabilizers as sodium laurilsulfate 0.8% and silica 0.2%. It furthermore has an average particle size of around 100 am. With this matrix-forming agent formulations of excellent fluidity and compressibility, good hardness values and low friability have been obtained, as a consequence of the polyvinylacetate's plasticity and the already known binding effect contributed by the polyvinylpyrrolidone.

With the object of masking unpleasant tastes soluble polymeric coatings have been used for instance, applied in amounts not less than 2% of the weight increase. Products based on hydroxipropylmethylcellulose as the Opadry and S 1 7003 or similar may be applied with this purpose. Another preferred product for masking tastes is found in the Eudragit E® of Röhm (copolymer of dimethyl aminoethyl methacrylate), which being soluble at pH under 5, avoids the drug release in the salivary pH. Other coatings used with this purpose consist in insoluble polymers used in low proportion or mixed with soluble polymers of the PVP type, for instance ethylcellulose (Aquacoat ECD 30®de FMC, Surelease® of Colorcon, Ethocel AQ® of Dow Chemical), neutral copolymers of esters of acrylic and methacrylic acid such as Eudragit NE-30D-Latex® of Röhm, copolymers of ethylacrylate, methylmethacrylate and trimethylaminomethacrylate (Eudragit RL/RL30D, Eudragit RS/RS30D® of Röhm).

Optionally, this invention may furthermore contain a film-forming coating with enteric coating applied on the slower sustained-release entity, as for instance the Kollicoat® MAE 100P or 30 DP of Basf. They consist of copolymers derivated from the methacrylic acid/ethylacrylate in a ratio of approximately 1:1, having an anionic character and sparingly acidic, an average molecular weight of approximately 250.000 and are vastly used in pharmaceutical products. Dissolving at pH over 5.5. The Kollicoat MAE 30 DP is marketed as an aqueous dispersion with 30% of solids, while the Kollidon MAE 100 P is a redispersable white powder.

Further examples of enteric coatings my be cellulose acetophtalate (CAP), Aquateric, cellulose acethyltrimellitate (CAT), hydroxipropylmethylcellulose phtalate: HP 50, HP 55 (dissolution at pH=5.0 and 5.5), succinic acid cellulose HPM: AqoatMF, AqoatHF, HPMC-ASLF, HPMC-ASMF, carboxymethyl and ethylethers of cellulose: Duodcel AQ, polyvinyl acetophtalate (PVAP): Opadry Oya/Oyp, Coateric, poly(maleic methyl vinylether-co-anhydrid): Gantrezan, copolymers of the methacrylic acid and methyl(or ethyl)methacrylate, Eudragit L100-55 pH=5.5, Eudragit L100 pH=6, Eudragit S pH=7 (both latter ones for release in more distal portions of the intestine), among others.

Preferably, the invention's pharmaceutical compositions is formulated in an oral dosage form, such as rigid capsule and/or tablet. And because resides it contains at least one excipient of pharmaceutical use.

This invention's composition may contain, besides those mentioned, other excipients of common use such as diluents, lubricants, binders and pH regulators, among others. Another aspect of the invention is related to a composition for oral administration comprising the hypnotic agent, together with one or more diluents, with one or more lubricants, with one or more binding agents, with one or more polymeric agents, with one or more pH regulators and with one or more coating agents.

The appropriate “hydrosoluble excipients” or “soluble or partially soluble diluentes” include DT lactose, mannitol, lactitol, saccharose, sorbitol, maltitol or pregelatinized starch, among others.

The appropriate “insoluble diluents or excipients” include microcrystalline cellulose, calcium phosphate, or other excipients based on cellulose, such as powder cellulose with monohydrated alpha lactose (cellactose 80 ® of Meggle), silicified microcrystalline cellulose (Prosolv® of JRS Pharma), among others.

The appropriate “lubricating agents” include magnesium stearate, stearic acid, calcium stearate, polyethylene glycols, hydrogenated vegetable oils and sodium stearyl fumarate, among others.

The additional conventional excipients which may be added include stabilizers, antioxidants, silica flow conditioners, bond breakers, or colors, among others.

Further diluents, lubricants, binders, coating agents and excipients which may be used are described in Handbook of Pharmaceutical Excipients, 2^{nd} Edition, American Pharmaceutical Association; The Theory & Practice of Industrial Pharmacy, 2^{nd} Edition, Lachman Leon, 1976; Pharmaceutical Dosage Forms: Tablets Volume 1, 2^{nd} Edition, Lieberman, Hebert A, et al, 1989; Modern Pharmaceutics, Banker, Gilbert and Rhodes, Christopher T, 1979; and Remington's Pharmaceutical Sciences, 15^{th} edition, 1975.

The previous compositions are completed at a final weight with excipients of pharmaceutical use and are dosed in rigid capsules or tablets are obtained which may be later coated with different purposes.

The invention's pharmaceutical composition may be prepared using common techniques and manufacturing processes generally known in the technique, as for instance dry-mixing the components.

Another object of the invention is the procedure to obtain the physical mixture between the Active and the rest of the composition's components of this invention:

### Obtaining the Core's Nucleus

The Zolpidem tartrate, the matrix-forming agent and the pH regulator were sieved through mesh Nr. 20.

The sieved powders were mixed together with an insoluble excipient.

The stearic acid lubricant previously sieved through mesh Nr. 60 was added to the previous blend and was mixed again.

The blend thus obtained was compressed in a rotary compressor at 80 mg average weight.

### Preparation and Application of the Core's Coating

The binder and the film former were added over a fraction of purified water, with mechanic agitation.

Polyvinylpyrrolidone was dissolved in another fraction of purified water and the pigments were added, recirculating the suspension in a colloid mill to reduce the solids particle's size.

The preparations of the previous steps were put together. With the resulting suspension, the inner nucleus were coated up to a theoretical weight increase of approximately 7 mg, testing the obtained gastroresistance.

### Obtaining the Outer Coating

The Zolpidem tartrate and the matrix-forming agent were sieved through mesh Nr. 20.

The sieved powders were mixed together with an insoluble excipient.

The stearic acid lubricant previously sieved through mesh Nr. 60 was added to the previous blend and was mixed again.

The blend obtained was compressed in a rotary compressor prepared for press-coating, as outer coating of an 260 mg average weight on the inner nucleus.

### Preparation and Application of the Outer Coating

A suspension at 13% P/P of the coating agent was prepared in purified water with the help of a mechanic agitator.

The tablets obtained were coated with press-coating with the prepared suspension, up to a theoretical weight increase of approximately 3 mg. testing the obtained gastroresistance.

The following examples show, but do not pretend to limit the different variants of the pharmaceutical compositions being appropriate to be used in this invention such as defined in this documentation. The examples defined are in now way restricting its total scope.

### EXAMPLE 1

For ZOLPIDEM LP 12.5 mg: coated tablets obtained by direct compression, where the matrix-forming polymer is the same in both entities.

Each coated tabled is composed, from inside to the outside, by:

| | Theoretical | | | |
|---|---|---|---|---|
| | weight | | | |
| | (per tablet) | | | |
| Nucleus | 80.000 | mg | | |
| | | | {close oversize brace} | Inner nucleus |
| Nucleus coating | 7.000 | mg | | |
| Outer layer | 260.000 | mg | | |
| | | | {close oversize brace} | Outer tablet |
| Outer coating | 3.000 | mg | | |
| Total | 350 | mg | | |

1. Quali-quantitative formula of the inner nucleus

Nucleus

| | Amounts per | | |
|---|---|---|---|
| Raw material | tablet | | % P/P |
| Zolpidem tartrate | 5.500 | mg | 6.875 |
| Hydrosoluble excipient | 44.500 | mg | 55.625 |
| Matrix-forming agent | 28.000 | mg | 35.000 |
| PH regulator | 0.400 | mg | 0.500 |
| Lubricant | 1.600 | mg | 2.000 |

Nucleus coating (enteric)

| | Amounts per | |
|---|---|---|
| Raw material | tablet | % P/P |
| Film former | 4.848 mg | 69.252 |
| Propylene glycol | 0.484 mg | 6.925 |
| Binder | 0.159 mg | 2.262 |
| Titanium dioxide | 0.226 mg | 3.232 |
| Talc | 1.283 mg | 18.329 |

2. Quali-Quantitative formula of the outer tablets

Outer layer

| | Amounts per | |
|---|---|---|
| Description | tablet | % P/P |
| Zolpidem tartrate | 7.000 mg | 2.692 |
| Hydrosoluble excipient | 182.800 mg | 70.308 |
| Matrix-forming agent | 52.000 mg | 20.000 |
| Insoluble excipient | 13.000 mg | 5.000 |
| Lubricant | 5.200 mg | 2.000 |

Outer coating

| | Amounts per | |
|---|---|---|
| Description | tablet | % P/P |
| Coating agent | 3.000 mg | 100.00 |

### EXAMPLE 2

For ZOLPIDEM LP 12.5 mg: coated tablets obtained by direct compression, where the matrix-forming polymer differs among the sustained-release entities.

Idem example 1, where the nucleus has the following composition:

Nucleus

| | Amounts per | |
|---|---|---|
| Raw material | tablet | % P/P |
| Zolpidem tartrate | 5.500 mg | 6.875 |
| insoluble excipient 1 | 32.900 mg | 41.125 |
| insoluble excipient 2 | 20.000 mg | 25.000 |
| Matrix-forming agent | 20.000 mg | 25.000 |
| Fumaric acid | 0.800 mg | 1.000 |
| Lubricant | 0.800 mg | 1.000 |

### Manufacturing Technique

1. The Zolpidem tartrate, and the fumaric acid were sieved through mesh Nr. 20.
2. The sieved powders were mixed together with the insoluble excipients 1 and 2 and the Matrix-forming agent
3. The lubricant previously sieved through mesh Nr. 60 was added to the previous blend and was mixed again.
4. The blend thus obtained was compressed in a rotary compressor at 80 mg average weight.

### EXAMPLE 3

Nucleus containing 7.5 mg Zolpidem tartrate, obtained by wet way granulation.

Nucleus

| | Amounts per | |
|---|---|---|
| Raw material | nucleus | % P/P |
| Zolpidem tartrate | 7.500 mg | 7.500 |
| Insoluble excipient | 55.500 mg | 55.500 |
| Binder | 5.000 mg | 5.000 |
| Matrix-forming agent | 30.000 mg | 30.000 |
| PH regulator | 1.000 mg | 1.000 |
| Lubricant | 1.000 mg | 1.000 |

### Manufacturing Technique

1. The Zolpidem tartrate, and the pH regulator were sieved through mesh Nr. 20.
2. The sieved powders were mixed together with the insoluble excipient and the agglutinating agent.
3. The previous blend was humidified with purified water.
4. The lubricant previously sieved through mesh Nr. 60 was added to the previous blend and was mixed again.
5. The wet granulate was dried at a temperature of 40-50° C. until the residual humidity of 2-3% was gauged through the mesh Nr. 18.
6. The lubricant previously sieved through mesh Nr. 60 was added to the dry and ground granulate and then mixed.
7. The blend thus obtained was compressed in a rotary compressor at 100 mg average weight.

### EXAMPLE 4

Composition of an outer layer with 10 mg Zolpidem tartrate, using Hydroxiethylcellulose as matrix-forming polymer

| | Amounts every | |
|---|---|---|
| Description | 300 mg | % P/P |
| Zolpidem tartrate | 10.000 mg | 3.333 |
| Hydrosoluble excipient 1 | 167.600 mg | 55.867 |
| Binder | 15.000 mg | 5.000 |
| Starch 1500 ® | 30.000 mg | 10.000 |
| Matrix-forming polymer | 60.000 mg | 20.000 |
| Insoluble excipient | 15.000 mg | 5.000 |
| Lubricant | 2.400 mg | 0.800 |

Starch 1500 is partially pregelatinized starch, having a binding effect and also provides lubrication to the mixture. It is partially hydrosoluble.

Natrosol: hydroxiethylcellulose, matrix-forming polymer, subject to erosion.

### Manufacturing Technique

1. The Zolpidem tartrate, the Hydrosoluble excipient 1 and the Natrosol were sieved through mesh Nr. 20.
2. The sieved powders were mixed together with the insoluble excipient and the starch 1500.
3. The lubricant previously sieved through mesh Nr. 60 was added to the previous blend and was mixed again.
4. The blend thus obtained was compressed in a rotary compressor adapted for press-coating, on a nucleus containing 5 mg Zolpidem tartrate.

### EXAMPLE 5

Outer coating colored to mask the bitter taste

| Description | % P/P |
|---|---|
| Film-forming polymer | 83.464 |
| Plasticizer | 1.990 |
| Bondbreaker | 4.950 |
| Titanium dioxide | 9.395 |
| Aluminic lacquer FD&C blue Nr. 1 | 0.200 |

### Preparation Technique

1. The film-forming polymer was added over a fraction of isopropyl alcohol.
2. In another fraction of isopropyl alcohol the plasticizer was dissolved and the bond breaker was added together with the lacquer and the titanium dioxide, recirculating the suspension in colloid mill to reduce the solids particle's size.
3. The preparations of the previous steps were put together and with the resulting solution, the obtained tablets were coated by any of the previous methods, up to theoretical weight increase of approximately 3%.

### EXAMPLE 6

### Sustained-Release Pellets Obtained by Coating

Each entity is composed by:

| | Theoretical | | |
|---|---|---|---|
| | weight | | |
| | (per dose) | | |
| Nucleus | 157.890 mg | | |
| | | {close oversize brace} | Faster sustained-release entity |
| Nucleus coating | 39.600 mg | | |
| Nucleus | 120.900 mg | | |
| | | {close oversize brace} | Slower sustained-release entity |
| Nucleus coating | 63.800 mg | | |
| Total | 382.190 mg | | |

Quali-quantitative formula of the faster sustained-release entity

Nucleus

| | Amounts per | |
|---|---|---|
| Raw material | tablet | % P/P |
| Zolpidem tartrate | 8.000 mg | 5.067 |
| PH regulator | 7.890 mg | 4.997 |
| Sugar | 55.000 mg | 34.834 |
| Corn starch | 60.000 mg | 38.001 |
| Talc | 7.000 mg | 4.433 |
| Polyvinylpyrrolidone | 20.000 mg | 12.667 |

Nucleus coating

| | Amounts per | |
|---|---|---|
| Raw material | tablet | % P/P |
| Insoluble coating polymer | 4.848 mg | 69.252 |
| Plasticizer | 0.484 mg | 6.925 |

Quali-quantitative formula of the slower sustained-release entity

Nucleus

| | Amounts per | |
|---|---|---|
| Description | tablet | % P/P |
| Zolpidem tartrate | 4.500 mg | 3.722 |
| PH regulator | 2.400 mg | 1.985 |
| Sugar | 45.000 mg | 37.221 |
| Corn starch | 50.000 mg | 41.350 |
| Talc | 4.000 mg | 3.309 |
| Polyvinylpyrrolidone | 15.000 mg | 12.407 |

Nucleus coating

| | Amounts per | |
|---|---|---|
| Description | tablet | % P/P |
| Insoluble coating polymer | 58.000 mg | 90.909 |
| Plasticizer | 5.800 mg | 9.091 |

### Manufacturing Technique

### Manufacturing the Agglutinating Solution

1. The povidone was added sprinkling it slowly over a fraction of purified water, under mechanic agitation, continuing with the same until its complete dissolution.
2. The remaining purified water was then added making up to the wanted volume.

### Obtaining the Zolpidem Nucleus

1. A mixture of Zolpidem, pH regulator and talc was ground in a hammer mill until obtaining an impalpable texture.
2. The sugar-starach's inert nucleus were added in a conventional pan, starting to run it.
3. The agglutinating solution was slowly atomized, alternating with the ground powders sprinkling.
4. The nucleus so obtained were dried in a static oven at 40-50° C., and were sieved through a mesh #16.

### Obtaining the Coating's Solution

1. The insoluble coating polymer was dispersed under agitation over an isopropylic alcohol fraction together with the plasticizer.
2. The remaining alcohol was then added to make up to volume.

### Obtaining the Coated Pellets

1. The nucleus containing the active were placed in a Glatt fluid bed equipment equipped with the Wurster system (Bottom Spray), coating the same working at a 45° C. temperature.

### EXAMPLE 7

The tablet's composition shows the same composition than that one which has been revealed in the example 1.

Nucleus

| | Amounts per | |
|---|---|---|
| Raw material | tablet | % P/P |
| Zolpidem tartrate | 2.250 mg | 2.812 |
| Insoluble excipient | 62.150 mg | 77.688 |
| Matrix-forming agent | 12.000 mg | 15.000 |
| PH regulator | 2.400 mg | 3.000 |
| Lubricant | 1.200 mg | 1.500 |

Nucleus coating

| | Amounts per | |
|---|---|---|
| Raw material | tablet | % P/P |
| Film former | 4.155 mg | 69.252 |
| Propylene glycol | 0.415 mg | 6.925 |
| Polyvinylpyrrolidone | 0.136 mg | 2.262 |
| Titanium dioxide | 0.194 mg | 3.232 |
| Talc | 1.100 mg | 18.329 |

Outer layer

| | Amounts per | |
|---|---|---|
| Description | tablet | % P/P |
| Zolpidem tartrate | 4.000 mg | 1.538 |
| Hydrosoluble excipient | 182.800 mg | 70.308 |
| Matrix-forming agent | 54.000 mg | 20.770 |
| Insoluble excipient | 15.000 mg | 5.770 |
| Lubricant | 5.200 mg | 2.000 |

Outer coating

| | Amounts per | |
|---|---|---|
| Description | tablet | % P/P |
| Coating agent | 3.000 mg | 100.00 |

There is not doubt that when this invention is put into practice, alteration may be introduced as regards some form and construction details, without this to imply getting away from the basic principles clearly substantiated in the following clauses of the claims.

1. A modified-release pharmaceutical composition containing, as the active agent, a short-acting hypnotic agent or a pharmaceutically acceptable salt thereof, comprising two sustained-release pharmaceutical entities, differentiated from each other by a different release rate of the active agent wherein the release of the active agent from one of the entities starts before the release of the active agent from the other entity.

2. A composition according to claim 1, wherein the active agent is selected from zaleplon, zopiclone or its enantiomers such as the R or S-zopiclone, triazolam, temazepam, brotizolam, alimemazine, indiplon and Zolpidem or a pharmaceutically acceptable salt thereof.

3. A composition according to claim 2, wherein the active agent is Zolpidem or a pharmaceutically acceptable salt thereof.

4. A composition according to claim 1, wherein the faster sustained-release entity starts releasing the active agent before the slower sustained-release entity.

5. A composition according to claim 1, wherein the slower sustained-release entity is comprised of a nucleus of a tablet obtained by press-coating or particles (pellets or tablets), included in a capsule or in the matrix of a tablet, optionally coated by one or more polymeric coatings.

6. A composition according to claim 5, wherein at least one of said polymeric coatings is soluble at a pH of more than 5.

7. A composition according to claim 5, wherein at least one of said polymeric coatings delays the release of the active agent from the nucleus or particles.

8. A composition according to claim 5, wherein the faster sustained-release entity comprises an outer layer applied over a nucleus by means of a press-coating process or particles (pellets or tablets) inside a capsule or a matrix of a tablet surrounding the pellets, optionally coated by one or more polymeric coatings.

9. A composition according to claim 8, wherein at least one of said coatings has the property of masking taste.

10. A composition according to claim 8, wherein at least one of said polymeric coatings delays the release of the active agent from the coating or particles.

11. A composition according to claim 4, wherein the faster sustained-release entity has a release rate of the active agent between 3 and 10 times slower than a conventional immediate release form containing the same active agent.

12. A composition according to claim 1, comprising 2 to 20 mg Zolpidem tartrate.

13. A composition according to claim 12, wherein the faster sustained-release entity comprises 1 to 16 mg, preferably 6 to 10 mg, most preferably 3 to 5 mg Zolpidem tartrate.

14. A composition according to claim 12, wherein the slower sustained-release entity comprises 1 to 10 mg, preferably 4 to 6 mg, most preferably 2 to 4 mg Zolpidem tartrate.

15. A composition according to claim 5, wherein the nucleus, particles, coating and/or matrix comprise at least one matrix-forming agent and does not contain any disintegrating agent.

16. A composition according to claim 15, wherein the faster sustained-release entity is present in the form of an outer layer which is applied by a press-coating process over the slower sustained-release entity which is present in the form of a nucleus obtained by compression.

17. A composition according to claim 16, wherein the matrix-forming agent present in at least one of the sustained-release entities is selected from polymeric agents or lipidic substances.

18. A composition according to claim 17, wherein the matrix-forming agent present in the faster sustained-release entity is subject to erosion.

19. A composition according to claim 17, wherein the matrix-forming agent present is selected from derivatives of cellulose or mixtures of polyvinylacetate and polyvinylpyrrolidone.

20. A composition according to claim 19, wherein the matrix-forming agent present is a mixture of polyvinylpyrrolidone and polyvinyl acetate.

21. A composition according to claim 1, wherein one or both sustained-release entities are obtained by a direct compression process.

22. A composition according to any of claim 15, wherein the matrix-forming agent is used in a concentration of 5-80 wt. %, preferably 10-50 wt. % in each of the sustained-release entities.

23. A composition according to claim 22, wherein the matrix-forming agent is used in the slower sustained-release entity in a concentration exceeding the concentration in the faster sustained-release entity.

24. A composition according to claim 1, wherein the slower sustained-release entity comprises insoluble and highly compressible diluents.

25. A composition according to claim 24, wherein the insoluble diluents comprise microcrystalline cellulose.

26. A composition according to claim 1, wherein the faster sustained-release entity comprises soluble or partly soluble diluents.

27. A composition according to claim 26, wherein the soluble or partially soluble diluents comprise lactose and pregelatinized corn starch.

28. A composition according to claim 1, containing, as excipients, one or more diluent agents, one or more lubricants, one or more matrix-forming agents, one or more binding agents and/or one or more coating agents.

29. A composition according to claim 28, containing, as a soluble excipient, lactose, mannitol, lactitol, saccharose, sorbitol, maltitol or pregelatinized starch.

30. A composition according to claim 29, wherein said composition contains lactose as a soluble excipient.

31. A composition according to claim 28, wherein said composition contains magnesium stearate as the lubricating agent.

32. A composition according to claim 5, wherein said composition contains a film-forming agent in the hydroxypropylmethylcellulose coating.

33. A composition according to claim 1 for oral administration.

34. A composition according to claim 1 being a rigid capsule or a tablet.
